(19) United States
(12) Patent Application Publication
(10) Pub. No.: US 2004/0043971 A1
(43) Pub. Date: Mar. 4, 2004

(54) Method of treating and preventing hyperparathyroidism with active vitamin D analogs

(75) Inventors: Richard B. Mazess, Madison, WI (US); Stephen A. Strugnell, Madison, WI (US); Joyce C. Knutson, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: Bone Care International, Inc., Middletown, WI

(21) Appl. No.: 10/385,327

(22) Filed: Mar. 10, 2003

Related U.S. Application Data
(63) Continuation-in-part of application No. 10/127,005, filed on Apr. 19, 2002.
Application No. 10/127,005 is a continuation-in-part of application No. 09/501,093, filed on Feb. 9, 2000, now Pat. No. 6,376,479.
Application No. 09/501,093 is a continuation-in-part of application No. 08/907,660, filed on Aug. 8, 1997, now abandoned.
Application No. 08/907,660 is a division of application No. 08/798,958, filed on Feb. 11, 1997, now Pat. No. 5,707,980.
Application No. 08/798,958 is a continuation of application No. 08/415,488, filed on Apr. 3, 1995, now Pat. No. 5,602,116.

(51) Int. Cl. A61K 31/59

(52) U.S. Cl. 514/167

(57) ABSTRACT

This invention relates to a method for treating or preventing hyperthyroidism secondary to chronic kidney disease by administering a sufficient amount of an active vitamin D analog utilizing a variety of effective treatment protocols.

### CROSS-REFERENCE TO RELATED APPLICATIONS

This application is a continuation-in-part of Ser. No. 10/127,005, filed Apr. 19, 2002, which is a continuation-in-part of U.S. patent application Ser. No. 09/501,093, filed Feb. 9, 2000, now U.S. Pat. No. 6,376,479, which is a continuation-in-part of U.S. patent application Ser. No. 08/907,660 filed Aug. 8, 1997, now abandoned, which is a divisional of U.S. patent application Ser. No. 08/798,958, filed Feb. 11, 1997, now U.S. Pat. No. 5,707,980, which is a continuation of U.S. patent application Ser. No. 08/415,488, filed Apr. 3, 1995, now U.S. Pat. No. 5,602,116.

### STATEMENT REGARDING FEDERALLY SPONSORED RESEARCH OR DEVELOPMENT

Not Applicable

### BACKGROUND OF THE INVENTION

This invention relates to a method for treating or preventing hyperparathyroidism associated chronic kidney disease by administering a sufficient amount of an active vitamin D compound utilizing effective treatment protocols.

Historically, it has long been known that vitamin D plays a critical role regulating calcium metabolism. The discovery of the active forms of vitamin D in the 1970's [Holick, M. F. et al., Proc. Natl. Acad. Sci. USA 68, 803-804 (1971); Jones, G. et al., Biochemistry 14, 1250-1256 (1975)] and active vitamin D analogues [Holick, M. F. et al., Science 180, 190, 191 (1973); Lam, H. Y. et al., Science 186, 1038-1040 (1974)], caused much excitement and speculation about the usefulness of these compounds in the treatment of bone depletive disorders.

Animal and early clinical studies examining the effects of these active vitamin D compounds suggested that such agents would be useful in restoring calcium balance. However, the best indicator of the efficacy of vitamin D compounds to prevent or treat depletive bone disorders is bone itself (or, in the case of renal osteodystrophy, serum levels of parathyroid hormone (PTH)) rather than calcium absorption or calcium balance. Certain clinical studies with 1α,25-dihydroxyvitamin D₃, and 1α-hydroxyvitamin D₃ indicate that the ability of these agents to restore lost bone mass or bone mineral content is dose-related. [See, Ott, S. M. and Chesnut, C. H., Annals of Int. Med.; 110:267-274 (1989); Gallagher, J. C. et al., Annals of Int. Med.; 113:649-655 (1990); Aloia, J. et al., Amer. J. Med. 84:401-08 (1988); and Shiraki, M. et al., Endocrinol. Japan 32, 305-315 (1985)].

These clinical studies also indicate that at the dosage ranges required for these agents to be truly effective, toxicity in the form of hypercalcemia and hypercalciuria becomes a major problem. Attempts to increase the amount of 1α,25-dihydroxyvitamin D₃ above 0.5 μg/day have frequently resulted in toxicity. At dosage levels below 0.5 μg/day, clinically significant effects on bone are rarely observed. [See, Jensen, G. F. et al., Clin. Endocrinol. 16, 515-524 (1982); Christiansen, C. et al., Eur. J. Clin. Invest. 11, 305-309 (1981)]. Doses of 2 μg/day of 1α-hydroxyvitamin D₃ (1α-(OH)D₃) were found to have efficacy in increasing bone mass in patients exhibiting senile osteoporosis [Sorensen, O. H. et al., Clin. Endocrinol. 7, 169S-175S (1977)]. Data from clinical studies in Japan, a population that has low calcium intake, indicate that efficacy is found with 1α-hydroxyvitamin D₃ when administered at 1 μg/day [Shiraki, M. et al., Endocrinol. Japan. 32:305-315 (1985); Orimo, H. et al., Bone and Mineral 3, 47-52 (1987)]. However, at 2 μg/day, toxicity with 1α-hydroxyvitamin D₃ occurs in approximately 67% of the patients, and at 1 μg/day this percentage is approximately 20%.

Thus, due to their toxicity, 1-hydroxylated vitamin D₃ compounds can only be administered at dosages that are, at best, modestly beneficial in preventing or treating loss of bone or bone mineral content. Indeed, Aloia et al., recommend that alternative routes of administration be sought that might avoid the toxicity problems and allow higher dosage levels to be achieved. [Aloia, J. et al., Amer. J. Med. 84:401-408 (1988)].

Despite reported toxicities of 1α-hydroxyvitamin D₃ and 1α,25-dihydroxyvitamin D₃, these two compounds remain the drugs of choice for treatment of many bone depletive diseases. Both 1α-hydroxyvitamin D₃ and 1α,25-dihydroxyvitamin D₃ have been studied and are clinically used in certain countries in Asia and Europe to treat osteoporosis [Gillespie, W. J., et al., Abstract, The Cochrane Library, issue 2, 2001; DeChant, K. L. and Goa, K. L., Drugs & Aging, 5(4):300-317 (1994); Ikeda, K and Ogata, E., Mechanisms of Aging & Development 116:103-111 (2000); Tanizawa, T., Osteoporos. Int. 9:163-170 (1999); Civitelli, R., Calcif. Tissue 57:409-414 (1995); Parfitt, A. M., Drugs 36:513-520 (1988); Thompson, S. P. et al., Brit. Edit. Soc. Bone Joint Surgery, 72:1053-1056 (1990); Sairanen, S. et al., Calcif. Tissue Int. 67:122-127 (2000); Haas, H. G., Horm. Metab. Res. 11:168-171 (1979); Tilyard, M. W. et al., New England J. Med. 326:357-362 (1992); Aloia, J. F. et al., Am. J. Med. 84:401-408 (1988); Avioli, L., Calcif. Tissue Int. 65:2392-294 (1999); Orimi, H. et al., Calcif. Tissue Int. 54:370-376 (1994); Sorensen, O. H. et al., Clinical Endocrinol. 7 (Suppl.): 169S-175S (1997)] Some studies suggest that active vitamin D, such as 1α-hydroxyvitamin D₃ and 1α,25-dihydroxyvitamin D₃, appears to be more effective than precursors, e.g., vitamin D, in treating, e.g., osteoporosis. These drugs appear to be most effective in those patients that have defective calcium absorption, e.g., in osteoporosis. Active vitamin D also appears to be more effective in treating 1α,25-dihydroxyvitamin D₃ resistance in target organs, decline in responsiveness to PTH inducement of 1α,25-dihydroxyvitamin D₃ synthesis, and lower production of 1α,25-dihydroxyvitamin D₃ especially with aging. [Zerwekh, J. E. et al., J. Clin. Endocrinol. Metab. 56:410-413 (1983); Nordin, B. E. C. et al., Calcif Tissue Int. 65:307-310 (1999); Morris, H. A. et al., Calcif Tissue Int. 49:240-243 (1991); Shiraishi, A. et al., Calcif Tissue Int. 65:311-316 (1999); Silverberg, S. J. et al., New England J. Med. 320(5):277-281 (1989); Francis, R. M., Calcif. Tissue Int. 60:111-114 (1997); Francis, R. M. et al., Osteoporosis Int. 6:284-290 (1996); Theiler, R. et al., Int. J. Vit. Nur. Res. 68:36-41 (1998)]

Both 1α-hydroxyvitamin D₃ and 1α,25-dihydroxyvitamin D₃ are approved for the treating and preventing of secondary hyperparathyroidism in end-stage renal disease, although both drugs are not currently approved in all major pharmaceutical markets.

The disease of hyperparathyroidism is a generalized disorder resulting from excessive secretion of parathyroid hormone by one or more parathyroid glands. The disease is characterized by elevated blood parathyroid hormone (PTH) levels and parathyroid glandular enlargement.

Hyperparathyroidism is subcategorized into primary, secondary and tertiary hyperparathyroidism. In primary hyperparathyroidism, the growth of the parathyroid glands is autonomous in nature, is usually due to tumors, e.g., parathyroid adenomas, and is presumably irreversible. Such adenomas typically do not exhibit vitamin D receptors and exhibit a resistivity to natural hormone form of vitamin D, i.e., calcitriol or 1,25-dihydroxyvitamin D₃. In secondary hyperparathyroidism, associated with, e.g., 1,25-dihydroxyvitamin D₃ deficiency and/or resistance, the parathyroid gland hyperplasia is typically adaptive owing to resistance to the metabolic actions of the hormone, and is presumably reversible. Secondary hyperparathyroidism occurs in patients with, e.g., kidney disease, osteomalacia, and intestinal malabsorption syndrome. Tertiary hyperparathyroidism is characterized by an autonomous proliferation state of the parathyroid glands with biological hyperfunction. Tertiary hyperparathyroidism can occur in patients with secondary hyperparathyroidism, wherein the reversible hyperplasia associated with secondary hyperparathyroidism converts to an irreversible growth defect, the enlarged tissue having vitamin D receptors. In all forms of hyperparathyroidism, bone abnormalities, e.g., the loss of bone mass or decreased mineral content, are common and kidney damage is possible. Hyperparathyroidism is thus also characterized by abnormal calcium, phosphorus and bone metabolism.

Secondary (and tertiary) hyperparathyroidism is a significant clinical problem associated with chronic kidney disease or renal insufficiency. Chronic kidney disease (CKD) is a worldwide public health problem. In the United States, it is estimated that 11% of the adult population has varying stages of chronic kidney disease, with about 4% of U.S. adults having less than half of the normal kidney function of a young adult. Further, the prevalence of end-stage renal disease (i.e., kidney failure) has more than doubled during the past decade. At present, end-stage renal disease afflicts an estimated 300,000 individuals, and that number is predicted to reach more than 600,000 individuals by 2010.

CKD is defined as either kidney damage or glomerular filtration rate (GFR) of less than 60 mL/min/1.73 m² for more than three months. The level of GFR is widely accepted as the best overall measure of kidney function in health and disease. CKD is now classified in stages based on estimated kidney function as measured by GFR. Stage 1 is defined as normal kidney function with some kidney damage and a GFR of ≧90 mL/min/1.73 m²; stage 2 involves mildly decreased kidney function with a mild decrease in GFR, i.e., a GFR of 60-89 mL/min/1.73 m². Stage 3 is defined as moderately decreased kidney function with a GFR of 30-59 mL/min/1.73 m². Stage 4 is defined as severely decreased kidney function with a GFR of 15-29 mL/min/1.73 m². Stage 5 is kidney failure with a GFR of <15-29 mL/min/1.73 m² or dialysis. Stage 5 is also known as end-stage renal disease (ESRD).

As noted above, secondary hyperparathyroidism is a common finding in patients with chronic kidney disease. It is established that the reduction of renal 1,25(OH)₂-vitamin D₃ synthesis is one of the principal mechanisms leading to the secondary hyperparathyroidism in these patients and it has been shown that calcitriol possesses direct suppressive action on PTH synthesis. Therefore, administration of calcitriol has been recommended for the treatment of secondary hyperparathyroidism in these patients. However, as described below, calcitriol has potent hypercalcemic effects giving it a narrow therapeutic window which limits its usage, especially at high doses.

In chronic kidney disease, there is a progressive loss of cells of the proximal nephrons, the primary site for the synthesis of the vitamin D hormones (collectively “1α,25-(OH)₂D”) from 25-hydroxyvitamin D₃ and 25-hydroxyvitamin D₂. In addition, the loss of functioning nephrons leads to retention of excess phosphorus which reduces the activity of the renal 25-hydroxyvitamin D-1α-hydroxylase, the enzyme which catalyzes the reaction to produce the D hormones. These two events account for the low serum levels of 1α,25-(OH)₂D commonly found in patients with moderate to severe chronic kidney disease.

Reduced serum levels of 1α,25-(OH)₂D cause increased, and ultimately excessive, secretion of PTH by direct and indirect mechanisms. The resulting hyperparathyroidism leads to markedly increased bone turnover and its sequela of renal osteodystrophy, which may include a variety of other diseases, such as, osteitis fibrosa cystica, osteomalacia, osteoporosis, extraskeletal calcification and related disorders, e.g., bone pain, periarticular inflammation and Mockerberg's sclerosis. Reduced serum levels of 1α,25-(OH)₂D also can cause muscle weakness and growth retardation with skeletal deformities (most often seen in pediatric patients).

Previous clinical studies utilizing hormonally active vitamin D drugs in end stage renal disease patients, i.e., the treatment of secondary hyperthyroidism, have focused on compounds derived from vitamin D₃. Use of 1α,25-(OH)₂D₃ and 1α-(OH)D₃ as replacement therapy seeks to treat or prevent renal osteodystrophy by treating or preventing hyperparathyroidism in end stage renal disease patients. As noted above, 1α,25-(OH)₂D₃ often causes toxic side effects (hypercalcemia and hyperphosphatemia) at dosages above 0.5 μg, especially when concomitantly administered phosphate binders, such as calcium compounds, are used to control serum phosphorus. Most patients respond to oral treatment doses of 0.5 to 1.0 μg/day or intravenous doses between 0.5 and 3.0 μg three times per week. As also described above, the other commonly used vitamin D drug is 1α-(OH)D₃ which often causes toxic effects at dosages over 1.0 μg/day, especially when used with phosphate binders. Most patients require treatment dosages of 1.0 μg/day or more. When either drug, 1α,25-(OH)₂D₃ or 1α-(OH)D₃, is administered in higher dosages, both efficacy and toxicity are found to increase. Thus, the hormonally active vitamin D₃ compounds are limited in their therapeutic usefulness due to their inherent toxicities.

Attempts to reduce the toxic side effects of active vitamin D₃, in the renal failure setting, have included administration of a low calcium dialysate with an ionized calcium concentration of 1.25 mM. However, it has been found that use of the low calcium dialysate has lead to higher serum PTH and phosphorus levels in patients who do not receive increased doses of oral calcium supplements as phosphate binders. When the dosages of calcium supplements and phosphate binders are increased, serum levels of phosphorus can be controlled, but the incidence of hypercalcemia rises markedly. Thus, there are many problems associated with the use of current vitamin D therapies for secondary hyperparathyroidism.

Notwithstanding these known problems with use of the hormonally active vitamin D₃ for hyperparathyroidism, there is a need for vitamin D compounds, derivatives or analogs, and treatment protocols that have low inherent toxicity and are effective even in low dose.

### BRIEF DESCRIPTION OF THE INVENTION

In one aspect, the present invention provides a method of treating, i.e., ameliorating or preventing, hyperparathyroidism associated with chronic kidney disease by lowering or maintaining low blood parathyroid hormone (PTH) levels in a patient suffering from the disease. The method includes administering to a subject in need thereof an amount of an active vitamin D analog sufficient to lower elevated or maintain lowered blood parathyroid hormone (PTH) levels, i.e., sufficient to suppress parathyroid activity.

Specifically, the present invention provides a method of lowering or maintaining lowered blood PTH in patients suffering from hyperparathyroidism secondary to chronic kidney disease which includes administering to these patients an effective amount of a vitamin D analog of formula (I), as described hereinbelow, to lower or maintain lowered the blood PTH level. The analog of formula (I) is any active vitamin D compound which has potent biological activity but low calcemic activity relative to the active forms of vitamin D₃. Such compounds include 1α-OH-vitamin D₂; 1α,24-(OH)₂-vitamin D₂; 1α,24(S)-(OH)₂-vitamin D₂; 1α-OH-vitamin D₄; 1α,24-(OH)₂-vitamin D₄ and 1α,24(R)-(OH)₂-vitamin D₄, suitably, 1α-OH-vitamin D₂, 1α,24-(OH)₂-vitamin D₂ and its (S) epimer, 1α,24(S)-(OH)₂-vitamin D₂. The analog of formula (I) is administered in a dosage amount of 0.5 μg to about 100 μg/week. As used herein, the term “vitamin D analog” is meant to refer to compounds having vitamin D hormonal bioactivity. It is also noted that a shorthand notation is often used for the D hormones, e.g., 1α-hydroxy vitamin D₂ may be referred to as 1α-OH-vitamin D₂ or simply 1α-OH-D₂.

In another aspect, the invention is a pharmaceutical composition having serum (or plasma) PTH lowering activity, which includes, in unit dosage form, an effective amount of a vitamin D analog which is 1α-OH-vitamin D₂; 1α,24-(OH)₂-vitamin D₂; 1α,∝(S)-(OH)₂-vitamin D₂; 1α-OH-vitamin D₄; 1α,24-(OH)₂-vitamin D₄; and 1α,24(R)-(OH)₂-vitamin D₄; suitably, 1α-OH-vitamin D₂, 1α,24-(OH)₂-vitamin D₂ and its (S) epimer, 1α,24(S)-(OH)₂-vitamin D₂; and a pharmaceutically acceptable excipient.

The treatment method of the present invention is an alternative to conventional therapy with 1α,25-(OH)₂ vitamin D₃ or 1α-OH-vitamin D₃; the method is characterized by providing an active vitamin D compound having equivalent bioactivity but much lower toxicity than these conventional therapies. This is true especially in the case where oral calcium-based phosphate binders are used concomitantly to control serum phosphorus. As such, the method addresses a long felt need in hyperparathyroidism therapy secondary to chronic kidney disease.

A fuller appreciation of the specific attributes of this invention will be gained upon an examination of the following detailed description of preferred embodiments, and appended claims.

### BRIEF DESCRIPTION OF THE DRAWING(S)

Not applicable.

### DETAILED DESCRIPTION OF THE INVENTION

The present invention relates to hyperparathyroidism secondary to chronic kidney disease (CKD) and to methods of ameliorating or preventing the disease by administering an effective amount of an active vitamin D analog utilizing a variety of treatment protocols. An elevated blood parathyroid hormone level, i.e., hyperparathyroidism, is typically associated CKD. Accordingly, the present invention will now be described in detail with respect to such endeavors; however, those skilled in the art will appreciate that such a description of the invention is meant to be exemplary only and should not be viewed as limitative on the full scope thereof.

More specifically, the present invention relates to therapeutic methods for lowering elevated blood levels of parathyroid hormone (PTH) which are secondary to CKD and/or maintaining lowered serum PTH levels. The method is of value in ameliorating or preventing hyperparathyroidism by administering an active vitamin D analog of formula (I), as described hereinbelow. The method in accordance with the present invention has significantly less resultant hypercalcemia and hyperphosphatemia, especially in patients who use oral calcium as a phosphate binder to control serum phosphorus levels. These attributes are achieved through a novel method of treating patients suffering from hyperparathyroidism associated with CKD.

In the following description of the method of the invention, process steps are carried out at room temperature and atmospheric pressure unless otherwise specified. As used herein, the term “chronic kidney disease” refers to stage 1 through stage 5 of kidney disease as measured by reduced glomerular filtration rate (GFR) and/or kidney damage. Also, as used herein, the term “hyperparathyroidism” refers to primary, secondary and/or tertiary hyperparathyroidism, and mixed states thereof.

It has been found that when the analogs of formula (I), described hereinbelow, are administered to patients with elevated serum (or plasma) parathyroid hormone levels, PTH concentration is lowered with significantly less hypercalcemia and hyperphosphatemia than is observed after the same amount of activated vitamin D₃ administered in previously known formulations and dosing regimens. Thus, the compounds of formula (I) have an improved therapeutic index relative to active vitamin D₃ analogs administered using conventional protocols.

It has been shown that 1α-hydroxyvitamin D₂ (1α-(OH)D₂) has the same biopotency as 1α-hydroxyvitamin D₃ (1α-(OH)D₃) and 1α,25-dihydroxyvitamin D₃ (1α,25-(OH)₂D₃) but is much less toxic [see, U.S. Pat. No. 5,403,831 and U.S. Pat. No. 5,104,864]. Even dosages up to 10 μg/day of 1α-(OH)D₂ in women with postmenopausal osteoporosis (in both open label and double blind testing) exhibited only mild hypercalciuria (>300 mg/24 hrs), and marked hypercalcemia (>11.0 mg/dL) solely due to 1α-(OH)D₂ was not evident. Additionally, 1α-(OH)D₂ did not adversely affect kidney function, as determined by creatinine clearance and BUN; nor did it increase urinary excretion of hydroxyproline, indicating the absence of any stimulatory effect on bone resorption. Administration of 1α-(OH)D₂ to healthy adult males in dosages up to 8 μg/day has shown no hypercalcemia or other adverse effects.

Compounds of this invention are useful in treating diseases caused by elevated levels of parathyroid hormone. In one aspect, compounds of the invention are used in treating hyperparathyroidism secondary to chronic kidney disease, and concomitantly, with reversing or reducing the bone loss associated with renal insufficiency. The patients so treated have GFRs<60 mL/min/1.73 m², and generally GFRs≧19-29 mL/min/1.73 m², suitably ≧30 mL/min/1.73 m².

The vitamin D analogs in accordance with the present invention have the general formula (I):

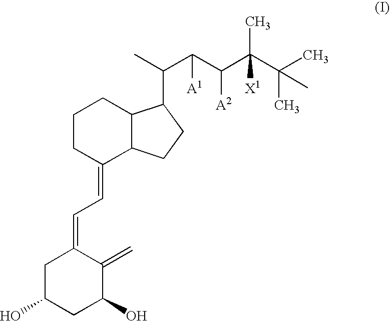

wherein A¹ and A² are each either hydrogen, or together represent a carbon-carbon double bond; and X¹ is either hydrogen or hydroxyl.

Further, for compounds of formulas (I) that have a chiral center, such as at the C-24 position, it is understood that both epimers (e.g., R and S) and the epimeric mixture are within the scope of the present invention. Where certain epimeric forms are preferred, the preferred form is substantially free of its other epimeric form, e.g., 1α,24(S)-dihydroxyvitamin D₂ is preferably substantially free of its (R) epimer, and 1α,24(R)-dihydroxy vitamin D₄ is preferred substantially free of its (S) epimer.

Such compounds in accordance with formulas I include generally 1α-hydroxyvitamin D compounds. Specific examples of such compounds of formulas (I) include, without limitation, 1α,24-dihydroxyvitamin D₂, 1α,24-dihydroxyvitamin D₄, specific epimeric forms such as 1α,24(S)-dihydroxyvitamin D₂ and 1α,24(R)-dihydroxy vitamin D₄, and include pro-drugs or pro-hormones such as 1α-hydroxyvitamin D₂, and 1α-hydroxyvitamin D₄.

The analogs of formula (I) are useful as active compounds in pharmaceutical compositions. The pharmacologically active analogs of this invention can be processed in accordance with conventional methods of pharmacy to produce pharmaceutical agents for administration to patients, e.g., in admixtures with conventional excipients such as pharmaceutically acceptable organic or inorganic carrier substances suitable for parenteral, enteral (e.g., oral), topical or transdermal application which do not deleteriously react with the active compounds. Suitable pharmaceutically acceptable carriers include, but are not limited to, water, salt (buffer) solutions, alcohols, gum arabic, mineral and vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc.

The pharmaceutical preparations can be sterilized and, if desired, mixed with auxiliary agents, e.g., lubricants, preservatives, stabilizers, wetting agents, emulsifiers, salts for influencing osmotic pressure, buffers, coloring, flavoring and/or aromatic active compounds. If a pharmaceutically acceptable solid carrier is used, the dosage form of the analogs may be tablets, capsules, powders, suppositories, or lozenges. If a liquid carrier is used, soft gelatin capsules, transdermal patches, aerosol sprays, topical creams, syrups or liquid suspensions, emulsions or solutions may be the dosage form.

For parenteral application, particularly suitable are injectable, sterile solutions, preferably oily or aqueous solutions, as well as suspensions, emulsions, or implants, including suppositories. Ampoules are convenient unit dosages. The dosage of the analogs for parenteral administration generally is about 0.5-30 μg given 1 to 3 times per week.

For enteral application, particularly suitable are tablets, dragees, liquids, drops, suppositories, or capsules such as soft gelatin capsules. A syrup, elixir, or the like can be used wherein a sweetened vehicle is employed.

Sustained or directed release compositions can be formulated, e.g., liposomes or those wherein the active compound is protected with differentially degradable coatings, such as by microencapsulation, multiple coatings, etc. It is also possible to freeze-dry the new compounds and use the lypolizates obtained, for example, for the preparation of products for injection. Transdermal delivery of pharmaceutical compositions of the analogs of formula (I) is also possible.

For topical application, there are employed as nonsprayable forms, viscous to semisolid or solid forms comprising a carrier compatible with topical application and having a dynamic viscosity preferably greater than water. Suitable formulations include, but are not limited to, solutions, suspensions, emulsions, creams, ointments, powders, liniments, salves, aerosols, etc., which are, if desired, sterilized or mixed with auxiliary agents, e.g., preservatives, etc.

Oral administration is preferred. Generally, the analogs of this invention are dispensed by unit dosage form comprising about 0.25 to about 10.0 μg in a pharmaceutically acceptable carrier per unit dosage. Suitably, an analog may be presented as 0.25 to 2.5 μg in unit dosage form. The dosage of the analogs generally is about 0.5 to about 100 μg per week, preferably about 0.5 μg to about 25 μg per week or 3.5 μg to 17.5 μg per week.

It is possible, if desired, to produce the metabolites of certain ones of the analogs of formula (I), in particular by nonchemical means. For this purpose, it is possible to convert them into a suitable form for administration together with at least one vehicle or auxiliary and, where appropriate, combined with one or more other active compounds.

The dosage forms may also contain adjuvants, such as preserving or stabilizing adjuvants. They may also contain other therapeutically valuable substances or may contain more than one of the compounds specified herein and in the claims in admixture.

As described hereinbefore, the analogs of formula (I) are preferably administered to the human patients in oral dosage formulation. As an analog in accordance with the present invention is released from the oral dosage formulation, and is absorbed from the intestine into the blood.

Those of ordinary skill in the art will readily optimize effective doses and co-administration regimens (as described hereinbelow) as determined by good medical practice and the clinical condition of the individual patient. Regardless of the manner of administration, it will be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, sex, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

Also included within the scope of the present invention is the co-administration of effective dosages of the analogs of formulas (I) in conjunction with hormones or other therapeutic agents, e.g., estrogens, which are known to ameliorate bone diseases or disorders typically associated with hyperparathyroidism. Such bone agents may include other vitamin D compounds, conjugated estrogens or their equivalents, calcitonin, bisphosphonates, calcium supplements, cobalamin, pertussis toxin and boron.

The term “co-administration” is meant to refer to a combination therapy by any administration route in which two or more agents are administered to a patient or subject. Co-administration of agents may be referred to as combination therapy or combination treatment. The agents may be in the same dosage formulations or separate formulations. For combination treatment with more than one active agent, where the active agents are in separate dosage formulations, the active agents can be administered concurrently, or they each can be administered at separately staggered times. The agents may be administered simultaneously or sequentially (i.e., one agent may directly follow administration of the other or the agents may be give episodically, i.e., one can be given at one time followed by the other at a later time, e.g., within a week), as along as they are given in a manner sufficient to allow both agents to achieve effective concentrations in the body. The agents may also be administered by different routes, e.g., one agent may be administered intravenously while a second agent is administered intramuscularly, intravenously or orally. In other words, the co-administration of the active vitamin D compound in accordance with the present invention with another therapeutic agent is suitably considered a combined pharmaceutical preparation which contains an active vitamin D compound and, e.g., a bone agent, the preparation being adapted for the administration of the active vitamin D compound on a daily or intermittent basis, and the administration of, e.g., a bone agent on a daily or intermittent basis. The agents also may be formulated as an admixture, as, for example, in a single tablet.

Possible dose ranges for exemplary co-administered agents are provided in Table 1.

TABLE 1
Possible Oral Dose Ranges for Various Agents Co-Administered
With Active Vitamin D Compounds of Formulas (I)-(IV)

| | Dose Ranges | | |
|---|---|---|---|
| | | | Most |
| Agent | Broad | Preferred | Preferred |
| Conjugated Estrogens or | 0.3-5.0 | 0.4-2.4 | 0.6-1.2 |
| Equivalent (mg/day) | | | |
| Sodium Fluoride (mg/day) | 5-150 | 30-75 | 40-60 |
| Calcitonin (IU/day) | 5-800 | 25-500 | 50-200 |
| Bisphosphonates (mg/day) | 50-2000 | 100-1500 | 250-1000 |
| Calcium Supplements | 250-2500 | 500-1500 | 750-1000 |
| (mg/day) | | | |
| Cobalamin (μg/day) | 5-200 | 20-100 | 30-50 |
| Pertussis Toxin (mg/day) | 0.1-2000 | 10-1500 | 100-1000 |
| Boron (mg/day) | 0.10-3000 | 1-250 | 2-100 |

Although the above dosages are for oral administration, it is understood that the co-administered agents can also be administered in alternative fashions, including intranasally, transdermally, intrarectally, intravaginally, subcutaneously, intravenously, and intramuscularly. It is also contemplated that some of the co-administered agents may be given on an other than daily basis.

Bulk quantities of the vitamin D analogs in accordance with the present invention can be readily obtained in accordance with the many widely known processes, e.g., as described in U.S. Pat. Nos. 3,907,843; 4,195,027; 4,202,829; 4,234,495; 4,260,549; 4,555,364; 4,554,106; 4,670,190; and 5,488,120; WO 94/05630, and Strugnell et al., 310 Biochem. J. 233-241 (1995), all of which are herein fully incorporated by reference.

The present invention is further explained by the following examples which should not be construed by way of limiting the scope of the present invention.

Comparison of 1α-OH-vitamin D₂ with 1α-OH-vitamin D₃

A comparison of 1α-(OH)D₂ to 1α-(OH)D₃ has been conducted. 1α-(OH) D₂ is equally active as 1α-(OH)D₃ in the healing of rickets, in the stimulation of intestinal calcium absorption and in the elevation of serum inorganic phosphorous of rachitic rats. [G. Sjoden et al., J. Nutr. 114, 2043-2946 (1984)]. In the same laboratory animal, 1α-OH-D₂ was found to be 5 to 15 times less toxic than 1α-OH-D₃ [see, also, G. Sjoden et al., Proc. Soc. Exp. Biol. Med. 178, 432-436 (1985)]. It has also now been found that, for example, 1α-OH-D₂ may be safely administered for up to two years to human subjects experiencing or having a tendency toward loss of bone mass or bone mineral content at dosages greater than 3 μg/day.

The following examples demonstrate that 1α-(OH)D₂ and 1α,24-(OH)₂D₄ are effective in reducing or preventing elevated blood PTH levels as well as preventing or restoring the loss of bone mass or bone mineral content while being substantially less toxic than 1α,25-(OH)₂D₃ and 1α-(OH)D₃. It is to be understood that although the following examples detail the use of 1α-(OH)D₂ and 1α,24-(OH)₂D₄, 1α,24(S)-(OH)₂D₂ may be readily utilized in the treatment of this invention with essentially equivalent results. For example, 1α,24(S)-(OH)₂D₂ shows activity equivalent to 1α,24(R)-(OH)₂D₃ and is also significantly less toxic than its vitamin D₃ counterpart.

### EXAMPLE 1

### Study Demonstrating Better Safety

The low toxicity of 1α-(OH)D₂ in human patients was demonstrated in a clinical study involving 15 postmenopausal osteoporotic women. [J. Bone Min. Res.; 9:607-614 (1994).] The selected patients were between 55 and 75 years of age, and exhibited L2-L3 vertebral bone mineral density (“BMD”) between 0.7 and 1.05 g/cm², as determined by measurements with a LUNAR dual-photon absorptiometer. (The mean bone mineral density in women with osteoporosis is about 0.85±0.17 g/cm², so that these limits correspond to about the 15th to 85th percentiles.)

On admission to the study, all patients received instruction on selecting a daily diet containing 400 to 600 mg of calcium. Compliance to this diet was verified at weekly intervals by 24-hour food records and by interviews with each patient.

All patients completed a one-week baseline period, a five- to seven-week treatment period, and a one-week post-treatment observation period. During the treatment period, patients orally self-administered 1α-(OH)D₂ at an initial dose of 0.5 μg/day for the first week, and at successively higher doses of 1.0, 2.0, 4.0, 5.0, 8.0 and 10.0 μg/day in each of the following weeks. All doses were administered before breakfast.

Blood and urine chemistries were monitored on a weekly basis throughout the study. Key blood chemistries included fasting serum levels of calcium, phosphorus, osteocalcin, creatinine and blood urea nitrogen. Key urine chemistries included 24-hour excretion of calcium, phosphorus and creatinine.

Data from the study clearly demonstrated that 1α-(OH)D₂ can be safely administered at high dose levels on a daily dosing regimen for periods of several weeks. In particular, the compound did not adversely affect kidney function, as determined by creatinine clearance and blood levels of urea nitrogen; nor did it increase urinary excretion of hydroxyproline, indicating the absence of any stimulatory effect on bone resorption. The compound had no effect on any routinely monitored serum chemistries, indicating the absence of adverse metabolic effects.

A positive effect of 1α-(OH)D₂ on calcium homeostasis was evident from dose-related increases observed in 24-hour urinary calcium levels, confirming that the compound increases intestinal calcium absorption, and from dose-related increases in serum osteocalcin, suggesting that the compound directly stimulates bone formation.

### EXAMPLE 2

### Study Demonstrating Safety and Efficacy for Human Osteoporosis

The safety and efficacy of 1α-(OH)D₂ as an oral treatment for osteoporosis was confirmed in a study involving 60 postmenopausal osteoporotic outpatients. The selected subjects had ages between 60 and 70 years, and exhibited L2-L3 vertebral BMD between 0.7 and 1.05 g/cm², as determined by dual-energy x-ray absorptiometry (DEXA). Exclusion criteria encompassed significant medical disorders and recent use of medications known to affect bone or calcium metabolism.

On admission to the study, each subject was assigned at random to one of two treatment groups; one group received up to a 104-week course of therapy with 1α-(OH)D₂; the other received only placebo therapy. All subjects received instruction on selecting a daily diet containing 700-900 mg of calcium and were advised to adhere to this diet over the course of the study. Compliance to the diet was verified at regular intervals by 24-hour food records and by interviews with each subject.

During the treatment period, subjects from one group orally self-administered 1α-(OH)D₂ at an initial dosage of 1.0 μg/day for one week, and increased the dosage to 2.0, 3.0, 4.0 μg/day in each of the following weeks, to a maximum dosage of 5.0 μg/day. The dosage for any given subject was increased in this way until the rate of urinary calcium excretion was elevated to approximately 275-300 mg/24 hours, at which point the subject held the dosage constant at the highest level attained. Subjects from the second group self-administered a matching placebo medication every day, titrating the apparent dosage upwards in the same manner as subjects being treated with 1α-(OH)D₂.

Spinal and femoral neck BMD were measured in all subjects by DEXA at the beginning of the study, and at six-month intervals thereafter. Intestinal calcium absorption was estimated in all subjects by a single isotope technique at the beginning of the study, and at 12-month intervals. Serum levels of vitamin D metabolites were determined by radioreceptor binding assays at baseline and at six-month intervals. Serum osteocalcin, serum PTH and urine hydroxyproline also were determined at baseline and at six-month intervals.

Other blood and urine chemistries were monitored at regular intervals during the treatment period. These chemistries included serum calcium, serum ionized calcium, urine calcium, blood urea nitrogen, serum creatinine and creatinine clearance. Kidney-ureter-bladder (KUB) x-rays were obtained at baseline and at 12-month intervals thereafter.

The results of the study are summarized below:

Subjects: Sixty subjects enrolled in what was originally intended to be a 52-week study. Of these 60 subjects, 55 completed one year of treatment (28 active; 27 placebo); and 41 subjects completed an optional second year of treatment.

Test Drug Dosages: The average prescribed dosage for subjects who received 1α-(OH)D₂ was 4.2 μg/day at 52 weeks and 3.6 μg/day at 104 weeks. The average prescribed dosage for placebo subjects was an apparent 4.8 μg/day at 52 weeks and 4.8 μg/day at 104 weeks.

Exclusions: One subject failed to comply with the prescribed dosage of test drug, as confirmed by an absence of serum 1α,25-(OH)₂D₂ at any time during the study. Data for this subject were excluded from analysis. Three patients were diagnosed with hyperparathyroidism when the PTH assays were completed (in batch) at the study's conclusion; data for these subjects were excluded from analysis. No subjects were excluded from analysis for noncompliance with the required dietary calcium intake of 700-900 mg/day.

Episodes of Hypercalcemia/Hypercalciuria: Marked hypercalcemia (>10.8 mg/dL) occurred in one subject in association with an intercurrent illness. The prescribed dosage of 1α-(OH)D₂ at the time of this episode was 5.0 μg/day. Moderate hypercalcemia (10.4-10.8 mg/dL) occurred in two subjects over the course of the study at prescribed dosages of 5.0 μg/day. Mild hypercalcemia (10.2-10.4 mg/dL) occurred in four subjects in the first year and in two subjects in the second year. Hypercalciuria was observed occasionally over the two-year study in 17 subjects treated with 1α-(OH)D₂.

Serum Calcium/Ionized Calcium: Mean serum calcium was approximately 0.1 to 0.2 mg/dL higher in subjects treated with 1α-(OH)D₂ than in subjects treated with placebo. This difference was significant (P<0.05) only during the second year of treatment. Mean serum ionized calcium was approximately 0.05 to 0.10 mg/dL higher in subjects treated with 1α-(OH)D₂.

Urine Calcium: Mean urine calcium increased during the initial titration period in a dose-response fashion. After titration, mean urine calcium was 50 to 130% higher (P<001) with 1α-(OH)D₂ treatment than with placebo treatment.

Kidney Function: No significant changes were observed with long-term 1α-(OH)D₂ treatment in BUN, serum creatinine or creatinine clearance. KUB x-rays revealed no abnormalities in either treatment group throughout the course of the study.

Bone: Bone mineral density (BMD) in the L2-L4 vertebrae progressively increased with 1α-(OH)D₂ treatment and decreased with placebo treatment over the two-year study. The difference in spinal BMD between the treatment groups became statistically significant (P<0.05) after 24 months of treatment. Similar changes were observed in femoral neck BMD with statistically significant differences observed after 18 months (P<0.001) and 24 months (P<0.05) of treatment.

Calcium Uptake: Intestinal absorption of orally administered ⁴⁵Ca increased by 40% (P<0.001) after 52 weeks of 1α-(OH)D₂ therapy, and by 29% (P<0.5) after 104 weeks of 1α-(OH)D₂ therapy, relative to placebo control.

Vitamin D Metabolites: Treatment with 1α-(OH)D₂ caused progressive increases in mean serum total 1α,25-(OH)₂D₃ from 21% (P<0.05) at six months to 49% (P<0.01) at 24 months relative to placebo therapy. This increase resulted from a dramatic rise in serum 1α,25-(OH)₂D₂ which was partially offset by a 50+% decrease in serum 1α,25-(OH)₂D₃. No treatment related changes were apparent in serum total 25-(OH)D.

Biochemical Parameters:

Serum levels of PTH decreased with 1α-(OH)D₂ therapy by 17% at 52 weeks and by 25% at 1-4 weeks, relative to placebo therapy.

Serum levels of osteocalcin were unchanged with long-term 1α-(OH)D₂ therapy.

Fasting urine hydroxyproline:creatinine ratio tended to decrease with long-term 1α-(OH)D₂ treatment but the observed differences between the 1α-(OH)D₂ and placebo treatment groups were not significantly different.

The results of this study clearly indicated that 1α-(OH)D₂ can be tolerated in higher long-term daily dosages than the commonly used vitamin D₃ analogues. They also showed that 1α-(OH)D₂ is well tolerated in postmenopausal women at long-term dosages in the range of 2.0 to 3.0 μg/day, provided that individuals exhibiting abnormally high urine calcium levels (when not receiving vitamin D therapy) are excluded from treatment. Long-term administration of such high dosages of 1α-(OH)D₂ significantly reduced bone loss at the spine and femoral neck, the most frequent sites of osteoporotic fractures. These positive effects on bone were accompanied by a sustained increase in intestinal calcium absorption and a sustained decrease in serum PTH. They were not accompanied by clear long-term trends in serum osteocalcin and urine hydroxyproline. Taken together, the results of this study demonstrate that 1α-(OH)D₂ is safe and effective in the treatment of postmenopausal or senile osteoporosis.

### Hyperparathyroidism Clinical Studies

### EXAMPLE 3

### Open Label Study in End Stage Renal Disease Patients Exhibiting Secondary Hyperparathyroidism

Five end stage renal disease patients were enrolled in an open label study. The selected patients had ages between 36 and 72 years and had been on hemodialysis for at least 4 months prior to enrollment. The patients each had an average serum phosphorus in the range of 3.0 to less than or equal to 6.9 mg/dL during the two months prior to enrollment (often controlled by oral calcium as a phosphate binder e.g., calcium carbonate or calcium acetate), and had a history of elevated serum PTH values of greater than 400 μg/mL when not receiving 1α,25-(OH)₂D₃ therapy.

Each patient had been receiving 1α,25-(OH)₂D₃ prior to enrollment, and discontinued the 1α,25-(OH)₂D₃ therapy for eight weeks prior to receiving 1α-(OH)D₂. After 8 weeks, the patients received treatment of 1α-(OH)D₂ at a dosage of 4 μg/day for 6 weeks. Throughout the eight-week washout period and the treatment period, patients were monitored weekly or biweekly for serum intact PTH level and weekly for excessive elevation in serum levels of calcium and phosphorus.

Throughout the washout period and treatment period, patients underwent routine hemodialysis (3 times per week) using a 1.25 mM calcium dialysate. They also ingested significant amounts of calcium as phosphate binders (1-10 g elemental Ca) to keep serum phosphorus levels below 6.9 mg/dL.

Average baseline values were as follows: serum PTH—480±21 μg/mL; serum Ca—8±0.3 mg/mL and serum phosphorus—5.1±0.2 mg/mL. In three patients, serum PTH decreased by 68%, 74% and 87% after two weeks. In the other two patients, serum PTH declined by 33% in one and 3% in the other after four weeks. Overall, serum PTH decreased by 49±17% and 33±9% after two and four weeks of 1α-OH-D₂, respectively, (p<0.05). Serum calcium (mg/dL) was 10.2±0.4 (p<0.05) and 9.8±0.2 (NS) and serum phosphorus (mg/dL) was 5.4±0.5 and 5.5±0.8 at two and four weeks, respectively (NS). A rise in serum PTH from the second to fourth weeks of 1α-(OH)D₂ treatment occurred when 1α-(OH)D₂ was withheld in three patients with serum PTH<130; they developed mild hypercalcemia (serum calcium, 10.3-11.4 mg/dL) that reversed after stopping 1α-(OH)D₂. No other adverse effects occurred. At 4-6 weeks of 1α-(OH)D₂ treatment of 4 μg, thrice weekly, four of five patients were in the target range of serum PTH; serum calcium was 10.0±0.2 mg/dL and serum phosphorus, 5.3±0.2 mg/dL. The patient who failed to respond to six weeks of 1α-(OH)D₂ treatment had a delayed response to both intravenous and oral calcitriol earlier, requiring several months of treatment before serum PTH fell. Serum PTH values in this patient fell by 38% after eight weeks of 1α-(OH)D₂ treatment. These data show that 1α-(OH)D₂ is efficacious and safe for the control of secondary hyperparathyroidism in end stage renal disease patients.

### EXAMPLE 4

### Double Blind Study of Bone in End Stage Renal Disease Patients

A twelve-month double-blind placebo-controlled clinical trial is conducted with thirty-five men and women with renal disease who are undergoing chronic hemodialysis. All patients enter an eight-week control period during which time they receive a maintenance dose of vitamin D₃ (400 IU/day). After this control period, the patients are randomized into two treatment groups: one group receives a constant dosage of 1α-(OH)D₂ (u.i.d.; a dosage greater than 3.0 μg/day) and the other group receives a matching placebo. Both treatment groups receive a maintenance dosage of vitamin D₃, maintain a normal intake of dietary calcium, and refrain from using calcium supplements. Oral calcium phosphate binders are used as necessary to maintain serum levels of phosphorus below 7.0 mg/dL. Efficacy is evaluated by pre- and post-treatment comparisons of the two patient groups with regard to (a) direct measurements of intestinal calcium absorption, (b) total body calcium retention, (c) radial and spinal bone mineral density, and (d) determinations of serum calcium and osteocalcin. Safety is evaluated by regular monitoring of serum calcium.

Analysis of the clinical data show that 1α-(OH)D₂ significantly increases serum osteocalcin levels and intestinal calcium absorption, as determined by direct measurement using a double-isotope technique. Patients treated with 1α-(OH)D₂ show normalized serum calcium levels, stable values for total body calcium, and stable radial and spinal bone densities relative to baseline values. In contrast, patients treated with placebo show frequent hypocalcemia, significant reductions in total body calcium and radial and spinal bone density. An insignificant incidence of hypercalcemia is observed in the treated group.

### EXAMPLE 5

### Double-Blind Study in End Stage Renal Disease (ESRD) Patients Exhibiting Secondary Hyperparathyroidism

Up to 120 ESRD (End Stage Renal Disease) patients undergoing chronic hemodialysis are studied in a multicenter, double-blind, placebo-controlled study. The selected patients reside in two major metropolitan areas within the continental U.S., have ages between 20 and 75 years and have a history of secondary hyperparathyroidism. They have been on hemodialysis for at least four months, have a normal (or near normal) serum albumin, and have controlled serum phosphorus (often by using oral calcium phosphate binders).

On admission to the study, each patient is assigned at random to one of two treatment groups. One of these groups receives two consecutive 12-week courses of therapy with 1α-(OH)D₂; the other receives a 12-week course of therapy with 1α-(OH)D₂ followed, without interruption, by a 12-week course of placebo therapy. Each patient discontinues any 1α,25-(OH)₂D₃ therapy for eight weeks prior to initiating 1α-(OH)D₂ therapy (4 μg/day). Throughout this eight-week washout (or control) period and the two subsequent 12-week treatment periods, patients are monitored weekly for serum calcium and phosphorus. Serum intact PTH is monitored weekly or biweekly, and bone-specific serum markers, serum vitamin D metabolites, serum albumin, blood chemistries, hemoglobin and hematocrit are monitored at selected intervals.

During the study, patients undergo routine hemodialysis (three times per week) using a 1.24 mM calcium dialysate and ingest calcium phosphate binders (such as calcium carbonate or calcium acetate) in an amount sufficient to keep serum phosphate controlled (6.9 mg/dL). Patients who develop persistent mild hypercalcemia or mild hyperphosphatemia during the treatment periods reduce their 1α-(OH)D₂ dosage to 4 μg three times per week (or lower). Patients who develop marked hypercalcemia or marked hyperphosphatemia immediately suspend treatment. Such patients are monitored at twice weekly intervals until the serum calcium or phosphorus is normalized, and resume 1α-(OH)D₂ dosing at a rate which is 4 μg three times per week (or lower).

During the eight-week washout period, the mean serum level of PTH increases progressively and significantly. After initiation of 1α-(OH)D₂ dosing, mean serum PTH decreases significantly to less than 50% of pretreatment levels. Due to this drop in serum PTH, some patients need to reduce their dosage of 1α-(OH)D₂ to 4 μg three times per week (or to even lower levels) to prevent excessive suppression of serum PTH. In such patients, exhibiting excessive suppression of serum PTH, transient mild hypercalcemia is observed, which is corrected by appropriate reductions in 1α-(OH)D₂ dosages.

At the end of the first 12-week treatment period, mean serum PTH is in the desired range of 130 to 240 μg/mL and serum levels of calcium and phosphorus are normal or near normal for end stage renal disease patients. For the placebo group, at the end of the second 12-week treatment period (during which time 1α-(OH)D₂ treatment is suspended and replaced by placebo therapy), mean serum PTH values markedly increase, reaching pretreatment levels. This study demonstrates that: (1) 1α-(OH)D₂-is effective in reducing serum PTH levels, and (2) 1α-(OH)D₂ is safer than currently used therapies, despite its higher dosages and concurrent use of high levels of oral calcium phosphate binder.

### EXAMPLE 6

### Open Label Study of Elderly Subjects with Elevated Blood PTH from Secondary Hyperparathyroidism

Thirty elderly subjects with secondary hyperparathyroidism are enrolled in an open label study. The selected subjects have ages between 60 and 100 years and have elevated serum PTH levels (greater than the upper limit of young normal range). Subjects also have femoral neck osteopenia (femoral neck bone mineral density of ≦0.70 g/cm 2).

Subjects are requested to keep a diet providing approximately 500 mg calcium per day without the use of calcium supplements. For a twelve week treatment period, subjects self-administer orally 2.5 μg/day 1α-(OH)D₂ At regular intervals throughout the treatment period, subjects are monitored for serum PTH levels, serum calcium and phosphorus, and urine calcium and phosphorus levels. Efficacy is evaluated by pre- and post-treatment comparisons of serum PTH levels. Safety is evaluated by serum and urine calcium and phosphorus values.

The administration of 1α-(OH)D₂ is shown to significantly reduce PTH levels with an insignificant incidence of hypercalcemia, hyperphosphatemia, hypercalciuria and hyperphosphaturia.

### EXAMPLE 7

### Double Blind Study of Elderly Subjects With Elevated Blood PTH from Secondary Hyperparathyroidism

A twelve month double-blind placebo-controlled clinical trial is conducted with forty subjects with secondary hyperparathyroidism. The selected subjects have ages between 60 and 100 years and have a history of secondary hyperparathyroidism. Subjects also have femoral neck osteopenia (femoral neck bone mineral density of ≦0.70 g/cm²).

All subjects enter a six-week control period after which the subjects are randomized into two treatment groups: one group receives a constant dosage of 15 μg/day 1α,24-(OH)₂D₄ (u.i.d.; a dosage greater than 7.5 μg/day), and the other group receives a matching placebo. Both groups maintain a normal intake of dietary calcium without the use of calcium supplements. Efficacy is evaluated by pre- and post-treatment comparisons of the two patient groups with regard to (a) intact PTH (iPTH); (b) radial, femoral and spinal bone mineral density; and (c) bone-specific urine markers (e.g., pyridinium crosslinks). Safety is evaluated by (a) serum calcium and phosphorus, and (b) urine calcium and phosphorus.

Analysis of the clinical data show that 1α,24-(OH)₂D₄ significantly decreases iPTH and bone specific urine markers. Subjects treated with this compound show normal serum calcium levels and stable radial and spinal bone densities relative to baseline values. In contrast, patients treated with placebo show no reduction in iPTH and bone-specific urine markers. An insignificant incidence of hypercalcemia is observed in the treatment group.

### EXAMPLE 8

### Open Label Study of Renal Patients With Sufficiently Elevated Blood PTH from Secondary and Tertiary Hyperparathyroidism

Fourteen renal patients enrolled in a clinical trial to study secondary hyperparathyroidism showed baseline iPTH levels greater than 1000 μg/mL (range: 10154706 μg/mL). These greatly elevated levels indicated a component of the disease as tertiary (i.e., glandular enlargement but continued presence of vitamin D receptors) to the gland as well as a component secondary to the loss of renal function. The initial dose of 1α-(OH)D₂ (10 μg-3 times/week) was increased (maximum, 20 μg-3 times/week) or decreased as necessary to attain and maintain iPTH in the range of 150-300 μg/mL. After 11-12 weeks of treatment, the iPTH levels of all but two of the patients had decreased to below 1000 μg/mL, and the iPTH levels in nine of the patients had decreased to below 510 μg/mL. There were no episodes of hypercalcemia with the patients during the study.

### EXAMPLE 9

### Placebo-Controlled Study of Subjects With Chronic Kidney Disease With Elevated Blood PTH

The safety and efficacy of 1α-(OH)D₂ (doxercalciferol) as a treatment for hyperparathyroidism associated with chronic kidney disease was confirmed in a study involving 55 adults, ages 18-85 years, with mild to moderate chronic kidney disease. The subjects had plasma intact parathyroid hormone (iPTH) levels above 85 μg/mL and completed an eight-week baseline period and then 24 weeks of therapy with either orally administered doxercalciferol or placebo.

The initial dose of test drug was 2 capsules daily (totaling 1.0 μg for subjects randomized to doxercalciferol treatment), with increases in steps of one capsule per day permitted after four weeks. The maximum dosage was limited to 10 capsules per day (5.0 μg/day of doxercalciferol). Subjects were monitored at regular intervals for plasma iPTH, serum calcium and phosphorus, 24-hour and fasting urinary calcium, bone-specific serum markers, plasma total 1α,25-(OH)₂D, and routine blood chemistries and hematologies. Glomerular filtration rate (GFR) was measured prior to beginning the treatment and at study termination. No physical or biochemical differences were detectable between the two treatment groups prior to starting treatment.

During doxercalciferol treatment, mean plasma iPTH progressively decreased from baseline levels, reaching maximum suppression of 45.6% after 24 weeks (p<0.001). No corresponding changes in mean iPTH were observed during placebo treatment. Mean iPTH was lower in subject receiving doxercalciferol versus placebo at all treatment weeks (p<0.001). No clinically significant differences in mean serum calcium, serum phosphorus and urine calcium or in rates of hypercalcemia, hyperphosphatemia and hypercalciuria were observed between treatment groups. Serum C- and N-telopeptides and bone-specific alkaline phosphate decreased with doxercalciferol treatment relative to baseline and placebo treatment (p<0.01). No differences between treatment groups were observed with regard to renal function and rates of adverse events. These data confirm that doxercalciferol can be used safely and effectively to control secondary hyperparathyroidism in chronic kidney disease patients.

The design of the study is summarized below.

Study Design: Pre-dialysis patients exhibiting secondary hyperparathyroidism associated with mild to moderate chronic kidney disease were recruited to participate in two multicenter, double-blinded, placebo-controlled studies conducted according to a common protocol. On enrollment, each subject was assigned, at random, in double-blinded fashion, to one of two treatment groups. Both treatment groups completed an 8 week Baseline Period (Weeks −8 to 0) and then underwent therapy with either orally administered doxercalciferol or placebo for a 24-week Treatment Period (Weeks 1 to 24). Irrespective of treatment group assignment, each subject discontinued any 1α,25-dihydroxivitamin D₃ (1α,25(OH)₂D₃) therapy for the duration of the study. Throughout the Baseline Period and the subsequent Treatment Period, subjects were monitored at regular intervals for plasma iPTH, serum calcium, serum phosphorus, and 24-hour and fasting urinary calcium, phosphorus and creatinine. Routine blood chemistries and hematologies, bone-specific serum markers, and plasma total 1α,25(OH)₂D were also monitored at selected intervals. Glomerular filtration rate (GFR) was measured prior to beginning treatment and at termination.

Subjects: Subjects qualified for inclusion in the Baseline Period if they were aged 18 to 85 years, had mild to moderate CR1 with serum creatinine between 1.8 to 5.0 mgldL (for men) or 1.6 to 4.0 mg/dL (for women), and had elevated plasma iPTH values (>85 μg/mL). Subjects receiving ongoing treatment with estrogen were required to maintain the same estrogen dosing regimen throughout the study. Subjects who began dialysis treatment or underwent renal transplantation were required to prematurely terminate participation. Screened patients were excluded if they had a current history of alcohol or drug abuse, were pregnant, possibly pregnant, or nursing, had a history of idiopathic urinary calcium stone disease, had undergone renal transplant surgery, or had received treatment in the past year with anticonvulsants, oral steroids, bisphosphonates, fluoride, or lithium. Patients were also excluded who had hypercalcemia, hyperthyroidism, sarcoidosis, malignancy requiring chemotherapy, hormonal therapy and/or radiation treatment, chronic gastrointestinal disease (i.e., malabsorption, surgery affecting absorption, and chronic ulcerative colitis), hepatic impairment, or any other condition which may have put the patient at undue risk. Qualified, enrolled subjects were precluded from entering the Treatment Period and prematurely terminated participation if they exhibited during the Baseline Period a urinary protein ≧4 grams/24 hours associated with a serum albumin ≦3.5 grams/dL, a urine calcium level (at Week −4) above 150 mg/24 hours, or a markedly elevated serum creatinine value (>5.0 mg/dL for men or >4.0 mg/dL for women).

Randomization: The two studies were conducted under double-blind conditions in each geographical region. Assignments of subjects to the two treatment groups were made randomly, by geographical region, in order of enrollment. The randomization was accomplished in subgroups of size 10, 5 subjects assigned to each of the two treatment groups. The randomization was performed by an independent statistician using the Statistical Analysis System (SAS).

Test Products: 1α-hydroxyvitamin D₂ (available as doxercalciferol from Bone Care International) was formulated for oral administration as soft elastic gelatin capsules in units of 0.5 mcg/capsule. Matching placebo capsules contained no doxercalciferol and were formulated from the same inactive ingredients in identical proportions. The inactive ingredients, in order of decreasing weight, were as follows: fractionated coconut oil, gelatin, glycerin, titaninum dioxide, FD&C Red #40, D&C Yellow #10, ethanol and butylated hydroxyanisole (BHA). Both active and placebo capsules were orange in appearance, imprinted with the logo “BCI,” and packaged in high-density polyethylene bottles, 50 capsules per bottle. The bottles were sealed with heat-induction tamper-evident seals and reusable child-resistant closures.

Dosing: The initial dose of test drug (doxercalciferol or placebo) was 2 capsules (totaling a 1.0 μg dose for subjects receiving doxercalciferol) every day before breakfast. This dosage was increased as necessary at monthly intervals, to suppress plasma iPTH levels by at least 30% from baseline. Dosage increases in steps of one capsule (0.5 μg) per day were permitted only if serum calcium was ≦9.6 mg/dL, serum phosphorus was ≦5.0 mg/dL, urine calcium was ≦200 mg/24 hours, and fasting urine calcium/urine creatinine ratio (urine Ca/Cr) was ≦0.25. The maximum dosage was limited to 10 capsules/day (5.0 μg/day of doxercalciferol or 35.0 μg/week).

Subjects suspended treatment if they developed moderate hypercalcemia (serum calcium ≧10.7 mg/dL corrected for serum albumin) and/or hypercalciuria (urine calcium >200 mg/24 hours or fasting urine Ca/Cr >0.25) during the Treatment Period. Such subjects were monitored weekly until the serum or urine calcium was normalized (≦10.2 mg/dL and/or ≦150 mg/24 hours or <0.25, respectively) and then resumed test drug dosing at a reduced rate with adjustment in their consumption of calcium-based phosphate binder, as appropriate. Subjects who developed mild hypercalcemia (serum calcium of 10.3 to 10.7 mg/dL) or hyperphosphatemia (serum phosphorus >5.0 mg/dL) during the Treatment Period adjusted their consumption of calcium-based phosphate binder and/or reduced their test drug dosage. At the discretion of the site Investigator(s), the dosage of calcium-based phosphate binder was increased for subjects who presented with hypocalcemia (≦9.0 mg/dL).

If one of the dosage levels was not optimum for a given subject (i.e., maintaining plasma iPTH suppression ≦30% from baseline and >15 μg/mL), the site Investigator(s) could vary the daily dosage administered according to a defined schedule (e.g., alternating dose of 1.0 μg with 0.5 μg) so that the total weekly dosage was optimized to the subject's needs.

Laboratory Procedures: Blood samples for analysis of serum chemistries, hematology and plasma iPTH were taken. Plasma iPTH samples were analyzed using a two-site immunoradiometric assay (IRMA).

The 24-hour urine samples for total protein and the 24-hour and spot urine samples for calcium, phosphorus, and creatinine were processed at the clinical sites. Urine samples for calcium, phosphorus and creatinine were acidified to a pH<2.0 using 6M HCL. Duplicate 4-mL aliquots of each urine sample were analyzed.

Blood samples for serum osteocalcin, bone-specific alkaline phosphatase, serum C-telopeptide (sCTx) and serum N-telopeptide (sNTx) were collected at the clinical sites. Triplicate 1-mL aliquots of serum from each sample were analyzed. All samples obtained from each subject for a given parameter were analyzed together in the same batch.

Blood samples for serum total 1α,25-dihydroxyvitamin D were analyzed. Serum samples from each subject were analyzed batchwise by means of radioreceptor assay following high-performance liquid chromatography.

GFR was determined at baseline and at termination by the Technetium or lothalmate (Glofill®) method. Each site used the same standardized method among all subjects at that study site. Serial blood and urine samples collected for GFR determination were analyzed on site or were sent on ice to the Cleveland Clinic in Cleveland, Ohio for analysis.

Data Treatment: Baseline values for all parameters were defined as the mean of the data collected during Weeks −4 and 0 of the Baseline Period. A positive response was defined as a reduction in mean plasma iPTH at Weeks 20 and 24 of ≧30% from baseline. At each time point, descriptive statistics were calculated, including n, mean, standard deviation, and standard error.

Also, the significance of the mean difference from baseline at each time point was assessed by paired t-test. This assessment was performed separately for each treatment group, with missing values being replaced by the last observat:ion carried forward (LOCF).

The treatment groups were compared at baseline and at each subsequent time point, and the significance of differences in means was assessed via two-sample t-test. For certain parameters, the data were recalculated as a percent of baseline and the analyses performed on these percentages instead of on the absolute data values.

All of the above analyses were performed on an intent-to-treat basis only, meaning that all subjects who received test drug were evaluated for statistical purposes. The protocol allowed for a per-protocol analysis that excluded subjects with low dosing compliance (<80% of prescribed doses). However, this analysis was not completed since compliance to the prescribed dosages was ≧80%, with few exceptions.

All adverse events, whether observed by staff or offered by subjects, were recorded, stating the type, onset, duration, severity, relationship to the study medication, and required treatment, and their frequency determined for each treatment group. For each type of serious, unexpected adverse event (SAE) or drug-related adverse experience, the treatment groups were compared with respect to the percent of subjects experiencing the adverse effect, by Fisher's exact test.

The results of the study are summarized below:

Patients Ineligible at Screening: One hundred thirty-three subjects were screened and 72 subjects (54%) entered the Baseline Period. The 61 screen failures were comprised of 28 patients with insufficiently elevated plasma iPTH levels (≦85 μg/mL), 9 patients with serum creatinine levels which were outside of the allowed range, 12 patients with both plasma iPTH levels ≦85 μg/ni and serum creatinine levels which were outside of the allowed range, three patients due to treatment with oral steroids, one patient due to treatment with anticonvulsants in the preceding year, one patient with a history of idiopathic renal stone disease, one patient who died prior to enrollment, five patients who declined to participate, and one patient who resided too far outside of the local area for 6 months during the year.

Discontinued Subjects: Seventy-two subjects were enrolled into the Baseline Period. Of the 72 enrolled subjects, 55 (76%) were admitted into the Treatment Period of the study. Seventeen subjects (24%) terminated or were disqualified during the Baseline Period and were precluded from entering the Treatment Period. Of these, eight subjects exhibited urine total protein levels ≧4 grams/24 hours associated with a serum albumin ≦3.5 grams/dL, three subjects had a markedly elevated serum creatinine (>5.0 mg/dL for men or >4.0 mg/dL for women) at either of the first two washout visits (Weeks −8 or −4), one subject demonstrated a serum creatinine level lower than that allowed by the inclusion criterion, three subjects declined to continue participating for personal reasons, and two experienced SAEs and were discontinued prematurely.

Nine subjects discontinued after entering and before completing the Treatment Period. One of the subjects relocated out of the area where the study was being conducted, one was found to have an intestinal malabsorption disorder, six experienced SAEs leading to discontinuation, and one experienced a non-serious adverse event leading to discontinuation.

Enrollment Demographics: The 55 subjects enrolled into the Treatment Period had physical and biochemical characteristics within the specified acceptable ranges and were otherwise qualified to participate in the study. These subjects had ages between 36 and 84 years (mean±SE 64.6±8.7 years). Forty-five subjects were men and 10 were women; 22 were African-Americans, 28 were Caucasians, four were Hispanics, and one was self-designated as “Other”. A comparison of the subjects assigned to active and placebo treatment with regard to physical and biochemical characteristics at baseline is provided in Table I. There were no statistically significant differences between these two groups for the tabulated characteristics.

Dosing Compliance: Dosing compliance was above 80% in 52 of the 55 treated subjects. Dosing compliance was 71% in one subject randomized to placebo treatment and 79% in another subject randomized to active treatment. A. third subject (active group) achieved only a 67% dosing compliance due to an adverse event unrelated to the drug. This subject discontinued participation in the study at Week 5.

Prescribed Dosages: The average (±SE) weekly prescribed dosages of test medication remained at the initial level of 2.0 capsules per day (1.0 mcg for subjects receiving doxercalciferol) for the first month, as required by the study protocol. Thereafter, the mean dose in the active group increased, reaching 3.28±0.39 capsules per day (1.61±0.20 mcg/day) by Week 24 (range: 1.0 to 3.5 meg/day). The mean dose in the placebo group also increased, reaching 5.13±0.49 capsules per day by Week 24 (range: 2.0 to 10.0 capsules/day). The mean weekly prescribed dose trended higher in the placebo group from Week 6 through Week 24, with the difference reaching statistical significance at Weeks 20 and 24.

Decreases in test drug dosage occurred in some subjects. The primary reason for a decrease in prescribed dose was suppression of plasma iPTH by more than 30% from baseline level. In a few cases, dosing with test medication was suspended for intercurrent illness and restarted, when possible, at the same level.

Clinical Laboratory Assessments: Laboratory data included in this report are limited to those specified in the protocol. In some cases, additional laboratory data were obtained in order to monitor adverse events or confirm previous determinations. There was significant variation in subject laboratory measurements during the Baseline Period as well as during the Treatment Period within and outside the laboratory normal reference ranges. Such variation is expected in the subjects who have CR1, since concomitant illness and complications related to renal disease are common. Laboratory abnormalities in individual subjects are not specifically discussed within this report unless attributed to the use of test medication or related to a serious adverse event.

Plasma iPTH At baseline, mean (±SE) plasma PTH was 219.1±22.3 pg/mL in the active group, with a range from 57 to 583 pg/mL and 171±14 pg/mL in the placebo group, with a range from 63 to 330 pg/mL. There was no difference in baseline iPTH levels between treatment groups (p=0.07). With initiation of doxercalciferol treatment, mean iPTH decreased to 165±15 pg/mL at Week 4 (p=O.O01 vs. baseline) and continued to decrease through Week 24, at which time the mean iPTH was 118±17 pg/mL (p<0.001 vs. baseline). In contrast, mean iPTH remained unchanged from baseline levels in the placebo group throughout the entire Treatment Period (p≧0.17), ending at 167±15 at Week 24. Mean iPTH was significantly lower in subjects receiving doxercalciferol at Weeks 16-24 (p<0.05 vs. placebo).

At the end of treatment, 20 (74%) of 27 subjects in the active group had achieved plasma iPTH suppression of ≧30% from baseline. This positive end-point response was based on the mean of plasma iPTH determinations at Weeks 20 and 24. Three of the other seven subjects had iPTH reductions of 24.0%, 24.2%, and 19.6%, respectively, and one subject had an increase in iPTH of 3.9%. The remaining three subjects showed the following responses: one discontinued participation in Week 17, at which time plasma iPTH was suppressed by 44.4%; another discontinued doxercalciferol treatment in Week 8, at which time plasma iPTH was suppressed by 27.9% from baseline; the third subject discontinued treatment in Week 5, at which time iPTH was increased by 22.8%. Only two (7.1%) of the 28 subjects treated with placebo achieved iPTH suppression of ≧30%.

Subjects randomized to doxercalciferol treatment exhibited progressively greater reductions in mean plasma iPTH during the course of the Treatment Period (see FIG. 1)˜Mean reduction of iPTH was 26.3% from baseline at Week 8, and 45.6% at Week 24. Mean iPTH reductions were significant (p<0.05 vs. baseline) from Week 2 through Week 24. Subjects randomized to placebo treatment exhibited no changes in mean plasma iPTH expressed as a percentage of baseline (p>0.17). Mean iPTH reduction was significantly greater in the active group at all Weeks except Week 6 (p<0.05).

Serum Calcium and Phosphorus Baseline mean (±SE) serum calcium level was 8.74±0.12 mg/dL in the active group and 8.82±0.13 mg/dL in the placebo group (NS). At Week 24, mean serum calcium was 9.14±0.11 mg/dL in the active group and 8.95±0.13 mg/dL in the placebo group (NS). The increase in mean serum calcium from baseline was significant (p<0.05) at Week 4 and at Weeks 12-24 in subjects treated with doxercalciferol, but not in subjects treated with placebo. Mean serum calcium differed between the treatment groups only at Week 20 (p<0.04).

At baseline, mean (±SE) serum phosphorus level was 4.02±0.15 mg/dL in the active group and 3.89±0.13 mg/dL in the placebo group (pNS). At Week 24, mean serum phosphorus was 4.27±0.13 mg/dL in the active group and 3.92±0.12 mg/dL in the placebo group (p=NS). The increases in mean serum phosphorus relative to baseline were not statistically significant in either treatment group, and mean serum phosphorus differed between groups only at Weeks 2 and 24 (p<0.05).

Two episodes of hypercalcemia (determined as corrected serum calcium>10.7 mg/dL) occurred in one subject receiving doxercalciferol treatment, with onsets in Week 4 and Week 16, respectively. The maximum serum calcium recorded during each of these episodes was 10.9 and 11.0 mg/dL, respectively, and the duration of each episode was 5 and 8 weeks, respectively. This subject had a serum calcium of 10.4 mg/dL at baseline and had exhibited serum calcium as high as 10.7 mg/dL during the Baseline Period. One episode of hypercalcemia (defined as corrected serum calcium >10.7 mg/dL) occurred in one subject receiving placebo treatment with onset in Week 12. The maximum serum calcium recorded during this episode was 10.9 mg/dL, and the duration of the episode was approximately 8 weeks. There were 9 episodes of hyperphosphatemia (defined as serum phosphorus >5.0 mg/dL) in 9 subjects during the Baseline Period. During the Treatment Period, there were 15 episodes of hyperphosphatemia in 10 subjects receiving active treatment and 9 episodes in 8 subjects receiving placebo treatment. Only one episode of Ca×P>65 occurred during the Treatment Period in one subject receiving placebo treatment.

Urine Calcium No statistically significant changes relative to baseline in mean 24 hour urine calcium or in mean fasting urine (Ca/Cr) were observed in either the active or placebo group throughout the Treatment Period. No differences between treatment groups reached statistical significance during the Treatment Period.

No episodes of hypercalciuria (defined as 24-hour urine calcium excretion greater than 200 mg or fasting urine Ca/Cr ratio above 0.25) occurred during the Treatment Period in either the active or placebo groups.

Renal Function—A rising trend in mean BUN and in mean serum creatinine relative to baseline was noted in both treatment groups, but changes from baseline were occasionally significant (p<0.05) only for the active group. However, no significant difference were observed between the groups during the Treatment Period.

GFR was measured at baseline and at the end of the study to compare the effects, if any, of active and placebo treatments on renal disease progression. Five subjects (18.5%) in the active treatment group and 8 subjects (28.6%) in the placebo group did not have a GFR measurement upon discontinuation or completion of the study. At baseline, mean GFR level was 33.5±3.0 (SE) mL/min in the active group and 36.9±3.3 mL/min in the placebo group. At Week 24, mean GFR was 29.7±3.0 mL/min in the active group and 35.1±3.3 mL/min in the placebo group. The difference in GFR between groups at Week 24 was not statistically significant (p 0.24).

Routine Chemistries and Hematologies Mean alkaline phosphatase was reduced significantly from baseline in the active group at Weeks 16 and 24 (p<0.05), but was not lowered in the placebo group during the Treatment Period. No other changes of clinical importance were observed from baseline or between groups for other routine laboratory parameters or in hematologies.

Serum Bone-Specific Markers and I a.25-dihydroxyvitamin D—Subjects treated with doxercalciferol showed mean reductions in serum bone-specific alkaline phosphatase (BSAP) from baseline of 19.7±3.7% by Week 16 (p<O.OI) and 27.9 t 4.6% by Week 24 (p<O.O I). Subjects treated with placebo showed no change in BSAP relative to baseline at any treatment week. Mean BSAP reductions differed significantly between treatment groups from Weeks 8 to 24 (p<O.OI). Similar reductions were observed in serum N- and C-telopeptides with doxercalciferol treatment. Mean serum osteocalcin trended upward from baseline with doxercalciferol treatment by nearly 10% at Week 4 and then progressively declined from baseline by about 20% at Week 24. Mean serum total 1 c˜,25-dihydroxyvitamin D levels increased significantly from baseline in the active group at all treatment weeks but did not differ significantly between groups at any treatment week.

Adverse Events—Twenty-seven SABs occurred in 17 subjects during the conduct of the studies. All of the SAEs were detennined to be unrelated to the test medication. Eighteen SAEs (67%) occurred when subjects were not being administered doxercalciferol. Three hundred fourteen (314) non-senous adverse events occurred during the conduct of both studies with 113 (36%) events occurring in subjects randomized to active treatment. One non-serious adverse event (0.3%), nausea of mild severity, reported in a subject who received doxercalciferol, was determined to be “possibly related” to the test medication. The remaining 313 non-serious events were determined to be “not related” to the test medication (95.6%), “probably not related” (3.5%), or “possibly related to another medicine” (0.6%). An analysis of the incidence rates for serious and non-serious adverse events by treatment group showed no significant differences.

Concomitant Medications—The most commonly prescribed medications, prescribed to more than 50% of the study subjects, included furosemide, calcium carbonate, warfarin, insulin (all types) and epoetin alfa. Thirty of the 55 subjects (54.5%) who entered the Treatment Period received a calcium-based phosphate-binding product.

Thus, the results demonstrated that during doxercalciferol treatment, mean plasma iPTH progressively decreased from baseline levels, reaching maximum suppression of 45.6% after 24 weeks (p<0.001), while no corresponding changes in mean iPTH were observed during placebo treatment. Mean iPTH was lower in subjects receiving doxercalciferol versus placebo at all treatment weeks (p<0.0001). No clinically significant differences in mean serum calcium, serum phosphorus and urine calcium or in rates of hypercalcernia, hyperphosphatemia and hypercalciuria were observed between treatment groups. Serum C- and N-telopeptides and bone-specific alkaline phosphate decreased with doxercalciferol treatment relative to baseline and placebo treatment (p<0.01). No differences between treatment groups were observed with regard to renal function and rates of adverse events. These results of this study demonstrate that doxercalciferol is safe and effective in the treatment of secondary hyperparathyroidism in CKD patients.

In summary, the present invention provides therapeutic methods for treating hyperparathyroidism associated chronic kidney disease. The methods are suitable for lowering elevated blood parathyroid hormone levels, or maintaining lowered blood PTH levels in subjects with hyperparathyroidism secondary to chronic kidney disease. The methods include administering an effective amount of an active vitamin D compound utilizing a variety of treatment protocols. The method in accordance with the present invention has significantly less resultant hypercalcemia and hyperphosphatemia.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

All patents, publications and references cited herein are hereby fully incorporated by reference. In case of conflict between the present disclosure and incorporated patents, publications and references, the present disclosure should control.

1. A method of lowering or maintaining lowered serum parathyroid hormone level in human patients suffering from hyperparathyroidism secondary to chronic kidney disease, comprising administering to the patients an effective amount of a vitamin D analog to lower and maintain lowered serum parathyroid hormone levels, the analog comprising a compound of formula (I): 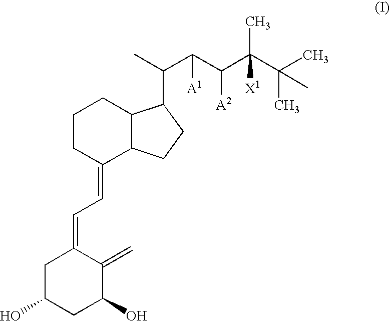
wherein A¹ and A² are each either hydrogen, or together represent a carbon-carbon double bond; and X¹ is either hydrogen or hydroxyl.

2. A method in accordance with claim 1 wherein the active vitamin D analog is 1α-(OH)-vitamin D₂, 1α,24-(OH)₂-vitamin D₂ or 1α,24(S)-(OH)₂-vitamin D₂.

3. A method in accordance with claim 2 wherein the active vitamin D analog is 1α-(OH)-vitamin D₂.

4. A method in accordance with claim 2, wherein the vitamin D analog is 1α,24-(OH)₂-vitamin D₂.

5. A method in accordance with claim 2, wherein the vitamin D analog is 1α,24(S)-(OH)₂-vitamin D₂.

6. A method in accordance with claim 1, wherein the patients have a glomerular filtration rate (GFR) of <60 mL/min/m².

7. A method in accordance with claim 1, wherein the patients have a glomerular filtration rate (GFR) of >15-29 mL/min/m².

8. A method in accordance with claim 1, wherein the patients have a glomerular filtration rate (GFR) of ≧30 mL/min/m².

9. A method in accordance with claim 1, wherein the chronic kidney disease is stage 1, stage 2, stage 3 or stage 4.

10. A method in accordance with claim 9, wherein the chronic kidney disease is stage 2 or stage 3.

11. A method in accordance with claim 1 wherein the amount of the vitamin D analog is administered parenterally or orally in combination with a pharmaceutically acceptable carrier.

12. A method in accordance with claim 11 wherein the amount of vitamin D analog is administered parenterally.

13. A method in accordance with claim 12 wherein the amount of vitamin D analog is administered intravenously.

14. A method in accordance with claim 11 wherein the amount of vitamin D analog is administered orally.

15. A method in accordance with claim 11 wherein the active vitamin D analog is co-administered with a phosphate binder.

16. A method in accordance with claim 12 wherein the active vitamin D compound is administered is by intravenous injection, nasopharyngeal or mucosal absorption, or transdermal absorption.

17. A method in accordance with claim 2 wherein the active vitamin D analog is administered in a weekly dosage of about 0.5 μg to about 100 μg.

18. A method in accordance with claim claim 2 wherein the active vitamin D analog is administered in a weekly dosage of about 0.5 μg to about 25 μg.

19. A method in accordance with claim 17, wherein the vitamin D analog is in a 0.5 μg per unit dosage form.

20. A method in accordance with claim 17, wherein the vitamin D analog is in a 2.5 μg per unit dosage form.

21. A method in accordance with claim 1, wherein the active vitamin D is co-administered with a calcium-based phosphate binder.

22. A method in accordance with claim 1, wherein the vitamin D analog is co-administered with at least one agent characterized by said agent's ability to reduce loss of bone mass, or bone mineral content in patients.

23. A method in accordance with claim 22, wherein the agent is other vitamin D compounds, conjugated estrogens, sodium fluorides, biphosphonates, cobalamin, pertussin toxin or boron.

24. A method in accordance with claim 22, wherein the vitamin D analog is administered before, after or concurrently with the other agent.

25. A method of treating hyperparathyroidism associated with chronic kidney disease, comprising administering to a subject suffering therefrom an amount of an active vitamin D analog which includes at least one of 1α-OH-vitamin D₂; 1α,24-(OH)₂-vitamin D₂; and 1α,24(S)-(OH)₂-vitamin D₂ sufficient to lower or maintain lowered blood parathyroid hormone (PTH) levels.

26. A method in accordance with claim 25, wherein the active vitamin D compound is 1α-OH-vitamin D₂.

27. A method in accordance with claim 25, wherein the active vitamin D compound is 1α,24-(OH)₂-vitamin D₂.

28. A method in accordance with claim 25, wherein the active vitamin D compound is 1α,24(S)-(OH)₂-vitamin D₂.

29. A method of treating hyperparathyroidism secondary to chronic kidney disease, comprising administering to a patient suffering therefrom an amount of 1α-OH-vitamin D₂ sufficient to lower or maintain lowered blood parathyroid hormone (PTH) levels.

30. A method in accordance with claim 29, wherein the patient has a glomerular filtration rate of <60 mL/min/m².

31. A method in accordance with claim 29, wherein the patients have a glomerular filtration rate (GFR) of >15-29 mL/min/m².

32. A method in accordance with claim 29, wherein the patients have a glomerular filtration rate (GFR) of ≧30 mL/min/m².

33. A method in accordance with claim 29, wherein the chronic kidney disease is stage 1, stage 2, stage 3 or stage 4.

34. A method in accordance with claim 33, wherein the chronic kidney disease is stage 2 or stage 3.
